(19) United States
(12) Patent Application Publication
(10) Pub. No.: US 2016/0271198 A1
(43) Pub. Date: Sep. 22, 2016

(54) Compositions and Methods for Promoting Sleep

(71) Applicant: Scientific Nutrition Products, Inc., Cambridge, MA (US)

(72) Inventors: Robert C. Jones, Belmont, MA (US); Bruce R. Bistrian, Ipswich, MA (US)

(21) Appl. No.: 14/922,949

(22) Filed: Oct. 26, 2015

Related U.S. Application Data
(63) Continuation-in-part of application No. 14/247,758, filed on Apr. 8, 2014.
(60) Provisional application No. 61/809,620, filed on Apr. 8, 2013.

(51) Int. Cl. A61K 36/736; A61K 31/405; A61K 45/06; A61K 47/36; A61K 36/42; A61K 47/12; A61K 31/4045; A61K 35/20

(52) U.S. Cl. CPC A61K 36/736; A23L 1/3051; A23L 1/3002; A61K 31/4045; A61K 31/405; A61K 35/20; A61K 47/36; A61K 36/42; A61K 47/12; A61K 45/06; A23V 2002/00

(57) ABSTRACT

Described herein are food formulations and methods, which promote sleep.

### CROSS-REFERENCE TO RELATED APPLICATIONS

This application is a continuation-in-part of U.S. application Ser. No: 14/247,758, filed Apr. 8, 2014, which claims priority to U.S. Provisional Application No. 61/809,620 filed Apr. 8, 2013. The entire contents of each of these applications are hereby incorporated by reference.

### BACKGROUND

All living organisms have intrinsic biological clocks or circadian rhythms that determine the timing of basic physiological occurrences, including the sleep-wake cycle. These rhythms cycle roughly every 24 hours and it is essential for the living being to synchronize its internal cycle to the external environment (daylight and nighttime, etc.) for well-being and survival. The brain essentially requires changes in autonomic and electrical activity roughly every 24 hours to maintain homeostasis in many domains. These changes are required to allow learning and memory to function ideally as well as manage brain energy balance in order to restore and rejuvenate brain cells.

In some animal models and humans, sleep cycles differ between the sexes due to gonadal hormones. However, both sexes are dependent on sleep for normal cognitive function, mental health and immune status. Indeed, when sleep cycles are not in synchrony with external light and dark cycles for long periods of time, humans develop disturbances such as emotional distress and lability, gastrointestinal dysfunction, and impaired immune status. Sleep deprivation has been used as a torture, and many days of sleep deprivation can lead to death of the organism.

Various studies have defined a normal sleep range as 7-8 hours per night. In these studies, “short sleep” is defined as habitual sleep time of 6 hours or less (Grander M A et al., Pack Sleep Medicine Reviews 2012; 14: 239-247; Dew M A et al., Psychosom Med 2003; 65(1):63-73). These studies have correlated an increase in mortality risk for those who sleep both less than and longer than the defined normal range. A comprehensive and systematic review of the literature has indicated that “short sleepers” (commonly less than seven hours per night, often less than five hours per night) have a 12% greater risk of mortality and “long sleepers” (commonly greater than eight or nine hours per night) a 30% greater risk of dying than those sleeping seven to eight hours per night. (Cappuccio, FP et al., Sleep 2008; 5:619-26).

“Short sleepers” are more likely to become chronically ill and to die prematurely, from coronary artery calcifications, cardiovascular risk predictors like hypertension, obesity, type 2 diabetes or impaired glucose control and atherogenic lipid profile. It is not clear why excessive sleep is also associated with an increase in mortality. However, associated factors such as depressive symptoms, low socioeconomic status, unemployment, low level of physical activity, undiagnosed health conditions, poor general health, and cancer-related fatigue may all contribute to mortality risk (Cappuccio, F P et al.)

The literature suggests that children 5 years of age require 12 hours per night, while adolescents require at least 9 hours per night. Elderly people, on the other hand, appear to need much less sleep. In a controlled study of 18 older (ages 60 to 76) and 35 younger (ages 18 to 32) healthy subjects, the older subjects slept for an average of 7.5 hours, while younger subjects slept an average of 9 hours (Klerman, E K and D Dijk Curr. Biol. 2008; 5:619-26).

It is estimated that sleep time has decreased in the general population in recent times by 2-3 hours per night (Webb W B et al., Bull Psychon Soc. 1975; 6:47-8). Insomnia is reported by approximately 50% of older adults who are generally dissatisfied with the quality of their sleep. In the US, the Center for Disease Control states, “Nationwide, an estimated 50 to 70 million people suffer from chronic sleep loss and sleep disorders.” (www.CDC.gov) According to data from the National Health Interview Survey, nearly 30% of adults reported an average of 6 hours or less of sleep per day in 2005-2007. In 2009, only 31% of high school students reported getting at least 8 hours of sleep on an average school night (www.CDC.gov). The CDC has called insomnia a public health epidemic.

The US leads the world in cases of insomnia (followed by Germany and England) and sleep problems add an estimated $15.9 billion to US national health care costs. Less sleep time is also documented in US children and adolescents (Lytle, L A et al, Obesity 2011: 19(2):324-31. Worldwide, at least 30% of adults and 70% of adolescents are not getting enough sleep.

Many professions necessitate shift work, and the Nurses' Health Study has documented that nurses who have done shift work for over 20 years have a 60% increase in the risk of type 2 diabetes.

Every year, 330 million people in the United States, en masse, shorten sleep by one hour in the spring as we shift to Daylight Savings Time. Interestingly, in the week after that shift, there is a 16-17% increase in motor vehicle accidents and fatal motor vehicle accidents that are alcohol related. There is also a 5% increase in myocardial infarction that following week. This phenomenon reverses in the fall when the US gains one hour of sleep, and the week following there is a 5% decrease in myocardial infarction incidence. This alteration in events following a one hour difference in sleep time illustrates how sensitive the body is to sleep alteration.

Given that children sleep on average two hours less than was the norm decades ago, it also suggests that children are at high risk for learning and memory issues, as well as early disease, due to lack of sleep.

This decrease in sleep time has also been associated with an increase in obesity as measured by body mass index (BMI) (Patel, SR Obesity Reviews 2009:10 (Suppl. 2): 61-68). The mechanisms by which sleep duration is linked to BMI or weight gain have yet to be elucidated. However, current studies are focused on the role of several hormones, such as leptin, ghrelin, peptide YY (PYY), cortisol and others, which are generated by the gut and adipose tissue. They have been found to be significantly different in those who sleep 7-8 hours per night, compared to those who sleep 6 hours or less per night.

In addition to hypertension, type 2 diabetes, and metabolic syndrome (any of which could lead to mortality), deficits in executive functioning, learning, and memory have also been linked with short sleep. (Grandner, M et al., Pack Sleep Medicine Reviews 2012; 14: 239-247). Impaired executive functioning can manifest as impaired decision making and risky behavior. Psychological impairment from sleep deprivation can manifest as depression, anxiety, and mood dysregulation. (Kilgore W D et al., J. Sleep Res Mar 2006 ; 15(1) :7-13.

Different types of studies have consistently shown a relationship between sleep and body mass index (BMI). However, as stated initially, this relationship differs between the sexes. Increased BMI is associated with decreased sleep duration in men, but there is a U-shaped relationship in women, such that high BMI is seen in women with short sleep and also with prolonged sleep duration (Littman, A J et al., Int Jobes March 2007; 31(3):466-75; Patel, SR Obesity Reviews 2009:10 (Suppl. 2): 61-68).

In these studies, the lowest BMI was reported at around 7.7 hours sleep duration per night. Sleep duration less than 7 hours has been shown to be associated with increased risk of weight gain in longitudinal studies of self-reported sleep durations. In fact, a study that collected data over 13 years reported that every extra hour increase in sleep duration is associated with a 50% reduction in risk of obesity (Hasler, G et al., Sleep June 2004; 27(4) 661-6. This report must be tempered by the U-shaped relationship of sleep and BMI, which may be confounded by depression, for example, in those with high BMI and long sleep duration.

Adults who sleep 5-7 hours per night or less are 30-80% more likely to develop Type 2 diabetes, cardiovascular disease, hypertension and premature death as compared to those who sleep 8 hours or more. Chronic sleep deprivation increases appetite, levels of pro-inflammatory cytokines in the blood, blood pressure, evening cortisol and insulin and blood glucose levels (McKeon-Cowdin R, BMC Com Alt Med 2011; November 8;11:109; Hotamisligil, G S Nature (2006) 444: 860-7).

The increase in cortisol that occurs after short sleep can cause an increase in heart rate and blood pressure, as cortisol is a stress hormone. In addition, elevations in cortisol have been shown to increase the risk of weight gain, specifically abdominal weight gain, which is more highly correlated with disease such as type 2 diabetes, and cardiovascular disease.

Among children and adolescents, less sleep is correlated with academic and behavioral problems, in addition to elevated blood pressure and early type 2 diabetes. Studies in younger populations have shown that sleep deprivation is both a neurobiological and physiological stressor that can cause metabolic derangements.

Inadequate sleep time has been found to be a risk factor for adolescent obesity (Lytle, L A et al., Obesity 2011: 19:324-31). BMI is also inversely associated with sleep duration in adults. Multiple studies have found this association, and a recent meta-analysis has corroborated this relationship (Patel, S R and F B Hu Obesity 2008: 3: 643-53). More importantly, the mechanism of action can be extrapolated (but not proven as yet) to be a combination of increased hunger, increased fatigue, altered thermoregulation, and perhaps even more opportunity to eat due to less sleep time. Studies have shown that hormonal changes—namely increases in ghrelin and decreases in leptin—in those who sleep less have been associated with increased hunger based on visual analogue scales. It has also been shown that less sleep time causes a decrease in core temperature, which, if chronically associated with less sleep time, can be another mechanism leading to weight gain (due to decreased resting energy expenditure). The hypothesis that less sleep time produces more opportunity to eat is intriguing, based on the habits of many obese persons who eat in the evening up until bed time. Food is generally restricted in the habits of these patients during the day, leading to a vicious cycle that can perpetuate later bedtime.

“Short sleep” has been associated with metabolic syndrome in women, but this association may have been confounded by the sleep disturbances that are often seen in the menopause (Choi, J K et al., J. Exp. Med. 2011 225(3):187-93). Menopause disrupts the sleep cycle and hormonal replacement can normalize this derangement. Menopause has also been associated with higher prevalence of depression and weight gain. The interrelationship among menopause, weight gain and sleep is therefore complex and associations can be misleading. It may be, in fact, that menopause leads to disruption in sleep which then leads to weight gain through multiple mechanisms including depression and mood disorder. There is also recent evidence that “short sleep” leads to increased levels of ghrelin and decreased levels of PYY, leading to increased hunger and decreased satiety (Taheri S et al., PloS Med 2004; 3: e62). It could be that several factors occurring at similar time points in the lifecycle of a woman (here, menopause) tend to promote weight gain, which makes it difficult to study one factor without controlling for the other variables.

There is a need for new compositions and methods to promote sleep.

### SUMMARY

Featured herein are food formulations, which when ingested by an individual (e.g., a mammal, such as a human) induces and/or maintains sleep in that individual. An appropriate formulation may provide an appropriate amount of certain fruit juices clinically demonstrated to promote sleep, an appropriate amount of L-tryptophan in food form to increase serotonin and melatonin production and/or an appropriate amount of a high glycemic index carbohydrate to foster tryptophan uptake into the brain. An appropriate formulation may have a total carbohydrate to protein ratio, which is less than or equal to about 10:1. An appropriate formulation may have a relatively high concentration of tryptophan provided as food as compared to other large neutral amino acids (i.e. leucine, isoleucine, valine, tyrosine and phenylalanine) An appropriate formulation may be in any physical state, e.g., a liquid, gas or solid.

Eligible fruit juices could include cherries, olive oil, tomatoes, and grapes with the preferred source from cherries. Appropriate cherries may be tart, (i.e., have a relatively low sugar content) or sweet but all will contain high glycemic index sugars. Examples include Montmorency and Jerte Valley cherries. The total amount of fruit juice in the formulation may be at least about 0.5 ounces (if concentrated) and less than about 10 fluid ounces. For example, the amount of tart cherry juice concentrate may be in the range of about 0.5 to about 2.0 ounces.

L-tryptophan may be provided in a food selected from the group consisting of: meats, seafoods, milks, cheeses, whey protein (including its various constituent peptides), vegetables, fruits (e.g., bananas) legumes (e.g., soy), whole grain foods, rices or nuts. In addition, proteins or peptides, which are rich in tryprophan, and preferably limited in leucine, may also be incorporated into the food formulation. The total amount of L-tryptophan in the formulation may be approximately 3 grams. For example, the amount of L-tryptophan in the formulation may be in the range of about 0.5 grams to about 3.0 grams.

High glycemic index carbohydrates may be provided, for example, in a food selected from the group consisting of: cherry juices but may include additional sources such as glucose, maltose, maltodextrin, potato, pretzel, a fruit, a grain, parsnip, white bread (wheat endosperm only), white rice (rice endosperm only), a corn flake or an extruded breakfast cereal.

An appropriate formulation should not include foods which contain an ingredient that negatively impacts or disrupts sleep, such as caffeine, alcohol, certain spices or high calorie, high sugar or fatty foods.

Sleep promoting food formulations may optionally include foods, that contain additional sleep promoting ingredients, such as zinc (e.g., pumpkin seeds, dark chocolate, garlic and sesame seeds); magnesium (e.g., bran (rice, wheat or oat); oatmeal; dried herbs (coriander, chives, spearmint, dill, sage, basil and savory); squash, pumpkin or watermelon seeds; dark chocolate; flax, sesame seeds or sesame butter; brazil nuts; sunflower seeds; almonds; cashews; molasses; or dry roasted soybeans); taurine (e.g., fish, meat, breast milk, sea algae and sea plants); 5-hydroxytryptophan (e.g., milk and dairy, meat, nuts, seeds, fish, fruit, vegetables, dark chocolate); potassium (e.g., bananas, potatoes, prune juice, plums, oranges, orange juice, tomatoes, spinach, sunflower seeds and almonds) and L-theanine (e.g., chamomile, green or black tea or bay bolete mushrooms).

Also featured are methods for inducing and/or promoting sleep in an individual comprising the step of: having the individual consume an appropriate amount of a formulation at an appropriate period of time in advance of when the individual would like to fall asleep. For example, the sleep promoting formulation may be taken at least 15, 30, 45, 60 or 75 minutes before an individual wishes to fall asleep.

In addition to consuming an appropriate formulation, an individual may undertake additional approaches towards attaining and/or maintaining sleep, such as: 1) ensuring that the area where the sleep is to occur is sufficiently dark; 2) reducing stress by use of techniques such as biofeedback, other meditative or relaxation techniques or massage; 3) avoiding strenuous exercise immediately prior to sleeping; 4) avoiding consumption of foods that contain sleep disturbing ingredients immediately prior to sleeping; and/or 5) increasing light exposure during the day time.

Food formulations described herein have been clinically demonstrated to promote sleep; are pleasant tasting or acceptable in taste when employed to improve efficacy and are relatively low in calories.

One aspect of the invention relates to a sleep promoting food formulation comprising an appropriate amount of: (a) melatonin; (b) high glycemic index carbohydrate; (c) a sleep-inducing ingredient; and (d)L-tryptophan; or any combination thereof.

In some embodiments, the total carbohydrate to protein ratio of the formulation is less than or equal to about 10:1.

In some embodiments, the formulation has a larger concentration of tryptophan than other large neutral amino acids.

In some embodiments, the formulation is selected from the group consisting of a liquid, solid, and powder.

In some embodiments, the formulation is a liquid.

In some embodiments, the sleep-inducing ingredient is provided by a food selected from the group consisting of: cherry juices from Montmorency, cherry juices from Jerte Valley cherries, and cherry juices other than from Montmorency and Jerte Valley cherries.

In some embodiments, the sleep-inducing ingredient is selected from cherry juices from Montmorency, cherry juices from Jerte Valley cherries, or combination thereof.

In some embodiments, the sleep-inducing ingredient is provided as red tart cherry juice concentrate (68 BRIX).

In some embodiments, the red tart cherry juice concentrate (68 BRIX) is provided from about 26.5% w/w to about 79.5% w/w.

In some embodiments, the red tart cherry juice concentrate (68 BRIX) is provided at about 53% w/w.

In some embodiments, the L-tryptophan is provided in a food selected from the group consisting of: a meat, poultry, seafood, milk, cheese, whey protein or whey protein peptide, vegetable, fruit, legume, whole grain food, rice, nut, protein rich in tryptophan, and peptide rich in tryptophan.

In some embodiments, the L-tryptophan is provided as whey protein or whey protein peptide; the whey protein or whey protein or peptide is at about 3.15% w/w to about 6.3% w/w.

In some embodiments, the whey protein or whey protein peptide is at about 4.2% w/w.

In some embodiments, the high glycemic index carbohydrate is provided in a food selected from the group consisting of: glucose, maltose, maltodextrin, potato, pretzel, a fruit, a grain, parsnip, a white bread, a white rice, a corn flake, and extruded cereal.

In some embodiments, the formulation does not contain caffeine, alcohol, high calorie food, high sugar food, or fatty food.

In some embodiments, the formulation additionally comprises an ingredient selected from the group consisting of zinc, magnesium, taurine, 5-hydroxytryptophan, potassium, L-theanine, xanthan gum, Reb A 99%, Symlife natural cherry pomegranate flavor, monk fruit juice concentrate, natural masking flavor, and citric acid.

In some embodiments, the ingredient is xanthan gum.

In some embodiments, the xanthan gum is provided at 0% w/w to about 0.3% w/w.

In some embodiments, the xanthan gum is provided at about 0.1% w/w.

In some embodiments, the ingredient is REB A 99%.

In some embodiments, the ingredient is REB A 99% is provided at 0% w/w to about 0.1% w/w.

In some embodiments, the ingredient is REB A 99% is provided at about 0.04% w/w.

In some embodiments, the ingredient is Symlife natural cherry pomegranate flavor.

In some embodiments, the Symlife natural cherry pomegranate flavor is provided at 0% w/w to about 1.8% w/w.

In some embodiments, the Symlife natural cherry pomegranate flavor is provided at about 0.9% w/w.

In some embodiments, the ingredient is monk fruit juice concentrate.

In some embodiments, the monk fruit juice concentrate is provided at 0% w/w to about 0.3% w/w.

In some embodiments, the monk fruit juice concentrate is provided at about 0.15% w/w.

In some embodiments, the ingredient is a natural masking flavor.

In some embodiments, the natural masking flavor is provided at 0% w/w to about 1.32% w/w.

In some embodiments, the natural masking flavor is provided at about 0.88% w/w.

In some embodiments, the ingredient is citric acid.

In some embodiments, the citric acid is provided at 0% w/w to about 0.32% w/w.

In some embodiments, the citric acid is provided at about 0.16% w/w.

In some embodiments, the formulation comprises red tart cherry juice concentrate (68 BRIX) is provided at about 53% w/w, Whey protein or whey protein peptide at about 4.2% w/w, xanthan gum at about 0.1% w/w, Reb A 99% at about 0.04% w/w, Symlife natural cherry pomegranate flavor at about 0.9% w/w, monk fruit juice concentrate at about 0.15% w/w, natural masking flavor at about 0.88% w/w, and citric acid at about 0.16% w/w.36. The formulation of claim 35, which further comprises adding water to about 40.57% w/w.

In some embodiments, the formulation of claim 1 further comprises adding water to about 40.57% w/w.

Another aspect of the invention relates to a method for promoting sleep and/or maintaining a sleep state in a subject comprising the step of: having the subject consume an appropriate amount of the formulation of present invention at an appropriate period of time in advance of when the subject would like to fall asleep.

In some embodiments, the appropriate period of time is selected from the group consisting of 15, 30, 45, 60 or 75 minutes prior to when the subject would like to fall asleep.

In some embodiments, the subject further sleeps in the dark, minimizes stress, refrains from strenuous exercise, avoids consumption of sleep disturbing ingredients immediately prior to when the subject would like to fall asleep, and receives ample daytime light exposure.

Another aspect of the invention relates to a method for treating a sleep disorder in a subject comprising the step of: having the subject consume an appropriate amount of the formulation of the present invention.

In some embodiments, the sleep disorder is selected from the group consisting of sleep-related breathing disorder, sleep apnea, insomnia, sleep-related movement disorder, parasomnias, central disorder of hypersomnolence, sleep deprivation, and restless legs syndrome.

In some embodiments, the methods comprise a formulation which comprises red tart cherry juice concentrate (68 BRIX) is provided from about 19.875 g/dose to about 59.625 g/dose.

In some embodiments, the methods comprise a formulation which comprises tart cherry juice concentrate (68 BRIX) is provided at about 39.75 g/dose.

In some embodiments, the methods comprise a formulation which comprises whey protein or whey protein peptide is provided at about 2.3625 g/dose to about 4.725 g/dose.

In some embodiments, the methods comprise a formulation which comprises whey protein or whey protein peptide is provided at about 3.15 g/dose.

In some embodiments, the methods comprise a formulation which comprises xanthan gum is provided at 0 g/dose to about 0.24 g/dose.

In some embodiments, the methods comprise a formulation which comprises xanthan gum is provided at about 0.08 g/dose.

In some embodiments, the methods comprise a formulation which comprises REB A 99% is provided at about 0.25 g/dose to about 0.045 g/dose.

In some embodiments, the methods comprise a formulation which comprises REB A 99% is provided at about 0.03 g/dose.

In some embodiments, the methods comprise a formulation which comprises Symlife natural cherry pomegranate flavor is provided at 0 g/dose to about 1.36 g/dose.

In some embodiments, the methods comprise a formulation which comprises Symlife natural cherry pomegranate flavor is provided at about 0.68 g/dose.

In some embodiments, the methods comprise a formulation which comprises monk fruit juice concentrate is provided at 0 g/dose to about 0.22 g/dose.

In some embodiments, the methods comprise a formulation which comprises monk fruit juice concentrate is provided at about 0.11 g/dose.

In some embodiments, the methods comprise a formulation which comprises natural masking flavor is provided at 0 g/dose to about 0.99 g/dose.

In some embodiments, the methods comprise a formulation which comprises natural masking flavor is provided at about 0.66 g/dose.

In some embodiments, the methods comprise a formulation which comprises citric acid is provided at 0 g/dose to about 0.24 g/dose.

In some embodiments, the methods comprise a formulation which comprises citric acid is provided at about 0.12 g/dose.

In some embodiments, the methods comprise a formulation which comprises red tart cherry juice concentrate (68 BRIX) is provided at about 39.75 g/dose, Whey protein or whey protein peptide at about 3.15 g/dose, xanthan gum at about 0.08 g/dose, Reb A 99% at about 0.03 g/dose, Symlife natural cherry pomegranate flavor at about 0.68 g/dose, monk fruit juice concentrate at about 0.11 g/dose, natural masking flavor at about 0.66 g/dose, and citric acid at about 0.12 g/dose.

In some embodiments, the methods comprise a formulation which comprises adding water to about 30.42 g/dose.

It is contemplated that all embodiments described herein, including those described under different aspects of the invention, can be combined with one another where not specifically prohibited. Further features and advantages will become apparent from the following Detailed Description and Claims.

### DETAILED DESCRIPTION

Food Formulations

Described herein are food formulations, which when ingested by an individual (e.g., a human or other mammal) promotes and/or maintains sleep in that individual. As used herein, food refers to any substance, which may be consumed by a subject to provide nutritional support for the body. A food may be in any state, including a solid, a liquid, powder, freeze-dried, or a gas. For example, a fruit may be in the solid form in which it exists in nature or as a fruit juice or extract. In addition, a food formulation may be comprised of multiple foods or components of foods, such as constituent proteins, peptides, carbohydrates, lipids etc.

As described herein, appropriate food formulations contain an appropriate amount of fruit juices clinically demonstrated to promote sleep, L-tryptophan and a high glycemic index carbohydrate. When ingested by an individual, the formulation induces and maintains sleep for an appropriate period of time (e.g., 6, 7, 8, 9, 10, 11 or 12 hours). The ratio of total carbohydrate to total protein in a food formulation may be less than or equal to about 10:1.

Melatonin, also known as N-acetyl-5-methoxy-tryptamine is a naturally occurring compound found in animals, plants and microbes. Melatonin can be present in a variety of foods, including cherries (especially tart cherries such as Montmorency or sweet cherries such as Jerte Valley cherries), olive oil, tomatoes, grapes, walnuts, grains and rices. The amount of melatonin in an appropriate food formulation may be at least about 0.3 mgs and less than about 10 mgs. For example, the amount of melatonin can be in the range of about 0.3 to about 1.0 mgs. When the melatonin is provided by tart cherries, it may be in the form of a juice concentrate. For example, the melatonin may be provided in a tart cherry juice concentrate, which may have enhanced soporific qualities due to additional integral components.

L-tryptophan is one of 22 standard, naturally occurring amino acids and is considered an essential amino acid in the human diet. L-tryptophan may be contained in the following foods: meats, seafoods, fruits (bananas), milks, cheeses, whey protein (including its various constituent peptides), vegetables, fruits (e.g., bananas) legumes (e.g., soy), whole grain foods, rices or nuts. In addition, proteins or peptides, which are rich in tryptophan, and preferably limited in leucine, may also be incorporated into the food formulation. The amount of L-tryptophan in an appropriate food formulation may be at least about 3 grams. The amount may be less than about 10, 9, 8, 7 or 6 grams.

“High glycemic index carbohydrate” refers to a carbohydrate, which when ingested by a subject breaks down relatively quickly during digestion and releases glucose rapidly into the subject's blood stream and elicits a prompt insulin response. For example the common sugars in fruit juices are glucose, sucrose, and fructose with decreasing sweetness, glycemic index, and insulin stimulation, in that order. Insulin then serves to reduce the branched-chain amino acid content of the plasma and thus alters the ration of tryptophan/branched chain amino acids. Since tryptophan and branched chain amino acids use the same transport system into the brain, improving the ration of tryptophan/branched chain amino acids increases brain transport of tryptophan. This effect then serves to extend the increased availability of tryptophan to support serotonin and melatonin production for up to 5 hours and supplements the initial effects of pre-formed melatonin. Serotonin promotes sleep by modulating the sleep wake cycle. High glycemic index is typically defined as 70 and above. Examples of foods having high glycemic index carbohydrates include glucose (GI=100), maltose, maltodextrin, potatoes, pretzels, parsnips, white breads (wheat endosperm only), white rices (rice endosperm only), corn flakes and extruded breakfast cereals.

An appropriate formulation should not include an ingredient that prevents sleep. Examples include: caffeine, alcohol, certain spices or foods, which contain a lot of calories, fat or sugar.

Appropriate formulations may optionally include additional sleep promoting ingredients, such as zinc (present, for example, in pumpkin seeds, dark chocolate, garlic and sesame seeds); magnesium (present, for example in brans (rice, wheat or oat); dried herbs (coriander, chives, spearmint, dill, sage, basil and savory); squash, pumpkin or watermelon seeds; dark chocolate; flax, sesame seeds or sesame butter; brazil nuts; sunflower seeds;

almonds; cashews; molasses; or dry roasted soybeans (edamame); taurine (e.g., fish, meat, breast milk, sea algae and sea plants); 5-hydroxytryptophan (e.g., milk and dairy, meat, nuts, seeds, fish, fruit, vegetables, dark chocolate); potassium (e.g., bananas, potatoes, prune juice, plums, oranges, orange juice, tomatoes, spinach, sunflower seeds and almonds) and L-theanine (e.g., chamomile, green or black tea or bay bolete mushrooms). taurine (e.g., fish, meat, breast milk, sea algae and sea plants); 5-hydroxytryptophan (e.g., milk and dairy, meat, nuts, seeds, fish, fruit, vegetables, dark chocolate); potassium (e.g., bananas, potatoes, prune juice, plums, oranges, orange juice, tomatoes, spinach, sunflower seeds and almonds) and L-theanine (e.g., chamomile, green or black tea or bay bolete mushrooms).

In some embodiments, the formulation may comprise ingredients selected from the group consisting of tart cherry juice concentrate, Whey protein or whey protein peptide, xanthan gum, Reb A 99%, Symlife flavors or Symlife natural cherry pomegranate flavor, monk fruit juice concentrate, natural masking flavor, and citric acid, and combinations thereof.

In some embodiments, the tart cherry juice concentrate may comprise red tart cherry juice concentrate (68 BRIX) from about 26.5% w/w to about 79.5% w/w. In some embodiments, the tart cherry juice concentrate may comprise red tart cherry juice concentrate (68 BRIX) at about 53% w/w. In some embodiments, the tart cherry juice concentrate may comprise red tart cherry juice concentrate (68 BRIX) from about 19.875 g/dose to about 59.625 g/dose. In some embodiments, the tart cherry juice concentrate may comprise red tart cherry juice concentrate (68 BRIX) at about 39.75 g/dose.

In some embodiments, the formulation may comprise whey protein or whey protein peptide, as the tryptophan source, at about 3.15% w/w to about 6.3% w/w. In some embodiments, the whey protein or whey protein peptide is at about 4.2% w/w. In some embodiments, the formulation may comprise whey protein or whey protein peptide, as the tryptophan source, at about 2.3625 g/dose to about 4.725 g/dose. In some embodiments, the whey protein or whey protein peptide is at about 3.15 g/dose.

In some embodiments, the formulation may comprise xanthan gum at 0% w/w to about 0.3% w/w. In some embodiments, the xanthan gum is at about 0.1% w/w. In some embodiments, the formulation may comprise xanthan gum at 0 g/dose to about 0.24 g/dose. In some embodiments, the xanthan gum is at about 0.08 g/dose.

In some embodiments, the formulation may comprise REB A 99% at 0% w/w to about 0.1% w/w. In embodiments, the REB A 99% is at about 0.04% w/w. In some embodiments, the formulation may comprise REB A 99% at about 0.25 g/dose to about 0.045 g/dose. In embodiments, the REB A 99% is at about 0.03 g/dose.

In some embodiments, the formulation may comprise Symlife natural cherry pomegranate flavor at 0% w/w to about 1.8% w/w. In some embodiments, the Symlife natural cherry pomegranate flavor is at about 0.9% w/w. In some embodiments, the formulation may comprise Symlife natural cherry pomegranate flavor at 0 g/dose to about 1.36 g/dose. In some embodiments, the Symlife natural cherry pomegranate flavor is at about 0.68 g/dose.

In some embodiments, the formulation may comprise monk fruit juice concentrate at 0% w/w to about 0.3% w/w. In some embodiments, the monk fruit juice concentrate is at about 0.15% w/w. In some embodiments, the formulation may comprise monk fruit juice concentrate at 0 g/dose to about 0.22 g/dose. In some embodiments, the monk fruit juice concentrate is at about 0.11 g/dose.

In some embodiments, the formulation may comprise a composition to improve the taste of the formulation. In some embodiments, the composition is a natural masking flavor. In some embodiments, the formulation may comprise natural masking flavor at 0% w/w to about 1.32% w/w. In some embodiments, the natural masking flavor is at about 0.88% w/w.

In some embodiments, the formulation may comprise natural masking flavor at 0 g/dose to about 0.99 g/dose. In some embodiments, the natural masking flavor is at about 0.66 g/dose.

In some embodiments, the formulation may comprise a basic or acidic solution to modulate the pH. In some embodiments, the acidic solution is citric acid. In some embodiments, the formulation may comprise citric acid at 0% w/w to about 0.32% w/w. In some embodiments, the citric acid is at about 0.16% w/w. In some embodiments, the formulation may comprise citric acid at 0 g/dose to about 0.24 g/dose. In some embodiments, the citric acid is at about 0.12 g/dose.

In some embodiments, the formulation is adjusted to 100% w/w with water. The water may be natural, spring, purified, or tap water. In some embodiments, the formulation may comprise water at about 10.06% w/w to about 70.35% w/w. In some embodiments, the water is at about 40.57% w/w. In some embodiments, the formulation may comprise water at about 7.555 g/dose to about 52.5125 g/dose. In some embodiments, the water is at about 30.42 g/dose.

In some embodiments, the formulation may further comprise preservatives or additives to increase the shelf-life of the formulation.

Methods

Also featured are sleep promotion methods using the formulations of the present invention as natural, herbal, and/or nutrient supplements. The formulations of the present invention do not contain drugs, over-the-counter or prescription; but may be used by individuals who are taking or prescribed with drugs.

A sleep promotion method may comprise the step of consuming an appropriate amount of a sleep promoting food formulation at an appropriate time in advance of going to bed. For example, the sleep promoting formulation may be taken at least 15, 30, 35, 60 or 75 minutes before an individual wishes to fall asleep.

In addition to consuming an appropriate formulation, an individual may take one or more other steps aimed at promoting or maintaining sleep, such as ensuring that the area where the sleep is to occur is sufficiently dark; stress reduction; avoiding strenuous exercise or consuming foods with sleep disturbing ingredients immediately prior to such time as an individual wishes to go to sleep. Ample daytime light exposure may also be beneficial.

In some embodiments, the present invention provides for a method for treating a sleep disorder in a subject comprising the step of: having the subject consume an appropriate amount of the formulations of present invention. In some embodiments, the sleep disorder is selected from the group consisting of sleep-related breathing disorder, sleep apnea, insomnia, sleep-related movement disorder, parasomnias, central disorder of hypersomnolence, sleep deprivation, and restless legs syndrome.

The invention, now being generally described, will be more readily understood by reference to the following example, which is included merely for purposes of illustration of certain aspects and embodiments and is not intended to limit the invention.

### EXEMPLIFICATIONS

### The Example: Method for Making a Sleep Promoting Formulation

| Ingredient | % w/w | g/dose |
|---|---|---|
| RED TART CHERRY JUICE CONC (68 BRIX) | 53 | 39.75 |
| WHEY PROTEIN or WHEY PROTEIN PEPTIDE | 4.2 | 3.15 |
| (TRYPTOPHAN SOURCE) | | |
| XANTHAN GUM | 0.1 | 0.08 |
| REB A 99% | 0.04 | 0.03 |
| SYMLIFE NATURAL CHERRY POMEGRANATE | 0.9 | 0.68 |
| FLAVOR 408676 | | |
| MONK FRUIT JUICE CONCENTRATE | 0.15 | 0.11 |
| NATURAL MASKING FLAVOR 2217792 | 0.88 | 0.66 |
| CITRIC ACID | 0.16 | 0.12 |
| WATER | 40.57 | 30.43 |
| TOTAL | 100 | 75.00 |

1—Combine cherry juice concentrate, xanthan gum, citric acid and water. Mix with heat to 70-70 degrees C.

2—Add Whey protein or Whey protein peptide powder and mix until uniform (no suspended particles)

3—Remove from heat

4—Add Reb A, monk fruit concentrate and flavors. Mix until uniform.
Although not wishing to be bound by theory, it would appear that the tart cherry juice provided in the formulation provides a ready source of sleep-promoting ingredients, possibly including melatonin, which promotes sleep in the initial 30 minutes after ingestion. The cherry juice also provides a source of high-glycemic index carbohydrates to enhance brain tryptophan uptake, which fosters serotonin and melatonin production. Tryptophan is also provided in food form in a special protein mixture. The combination further facilitates the transport of tryptophan across the blood: brain barrier. Via a series of well-established pathways, the tryptophan converts to serotonin, which improves sleep quality and quantity, which further converts to melatonin over several hours, thereby providing a second wave of melatonin, which promotes sleep several hours after falling asleep. The formulation with all food ingredients helps one to fall asleep fairly rapidly and stay asleep for a sufficient period of time.

### REFERENCES

All publications and patents mentioned herein, including those references listed below, are hereby incorporated by reference in their entirety as if each individual publication or patent was specifically and individually incorporated by reference. In case of conflict, the present application, including any definitions herein, may control.

### EQUIVALENTS

Those skilled in the art will recognize, or be able to ascertain using no more than routine experimentation, many equivalents to the specific embodiments of the invention described herein. While specific embodiments of the subject invention have been discussed, the above specification is illustrative and not restrictive. Many variations of the invention may become apparent to those skilled in the art upon review of this specification. The full scope of the invention should be determined by reference to the claims, along with their full scope of equivalents, and the specification, along with such variations. Such equivalents are intended to be encompassed by the following claims.

1. A sleep promoting food formulation comprising an appropriate amount of:
(a) melatonin;
(b) high glycemic index carbohydrate;
(c) a sleep-inducing ingredient; and
(d)L-tryptophan;
or any combination thereof.

2-5. (canceled)

6. The formulation of claim 1, wherein the sleep-inducing ingredient is provided by a food selected from the group consisting of: cherry juices from Montmorency, cherry juices from Jerte Valley cherries, and cherry juices other than from Montmorency and Jerte Valley cherries.

7. The formulation of claim 6, wherein the sleep-inducing ingredient is selected from cherry juices from Montmorency, cherry juices from Jerte Valley cherries, or combination thereof.

8. The formulation of claim 7, wherein the sleep-inducing ingredient is provided as red tart cherry juice concentrate.

9. The formulation of claim 8, wherein the red tart cherry juice concentrate is provided from about 26.5% w/w to about 79.5% w/w.

10. The formulation of claim 9, wherein the red tart cherry juice concentrate is provided at about 53% w/w.

11-15. (canceled)

16. The formulation of claim 1, which additionally comprises an ingredient selected from the group consisting of zinc, magnesium, taurine, 5-hydroxytryptophan, potassium, L-theanine, xanthan gum, natural cherry pomegranate flavor, monk fruit juice concentrate, natural masking flavor, and citric acid.

17-34. (canceled)

35. The formulation of claim 1, comprises red tart cherry juice concentrate is provided at about 53% w/w, Whey protein or whey protein peptide at about 4.2% w/w, xanthan gum at about 0.1% w/w, natural cherry pomegranate flavor at about 0.9% w/w, monk fruit juice concentrate at about 0.15% w/w, natural masking flavor at about 0.88% w/w, and citric acid at about 0.16% w/w.36. The formulation of claim 35, which further comprises adding water to about 40.57% w/w.

36. (canceled)

37. A method for promoting sleep and/or maintaining a sleep state in a subject comprising the step of: having the subject consume an appropriate amount of the formulation of claim 1 at an appropriate period of time in advance of when the subject would like to fall asleep.

38. The method of claim 37, wherein the appropriate period of time is selected from the group consisting of 15, 30, 45, 60 or 75 minutes prior to when the subject would like to fall asleep.

39. The method of claim 38, wherein the subject further sleeps in the dark, minimizes stress, refrains from strenuous exercise, avoids consumption of sleep disturbing ingredients immediately prior to when the subject would like to fall asleep, and receives ample daytime light exposure.

40. The method of claim 37, wherein the formulation comprises red tart cherry juice concentrate is provided from about 19.875 g/dose to about 59.625 g/dose.

41. The method of claim 40, wherein the red tart cherry juice concentrate is provided at about 39.75 g/dose.

42-55. (canceled)

56. The method of claim 37, wherein the formulation comprises red tart cherry juice concentrate is provided at about 39.75 g/dose, Whey protein or whey protein peptide at about 3.15 g/dose, xanthan gum at about 0.08 g/dose, natural cherry pomegranate flavor at about 0.68 g/dose, monk fruit juice concentrate at about 0.11 g/dose, natural masking flavor at about 0.66 g/dose, and citric acid at about 0.12 g/dose.

57. (canceled)

58. A method for treating a sleep disorder in a subject comprising the step of: having the subject consume an appropriate amount of the formulation of claim 1.

59. The method of claim 58, wherein the sleep disorder is selected from the group consisting of sleep-related breathing disorder, sleep apnea, insomnia, sleep-related movement disorder, parasomnias, central disorder of hypersomnolence, sleep deprivation, and restless legs syndrome.

60. The method of claim 58, wherein the formulation comprises red tart cherry juice concentrate is provided from about 19.875 g/dose to about 59.625 g/dose.

61. The method of claim 60, wherein the tart cherry juice concentrate is provided at about 39.75 g/dose.

62-75. (canceled)

76. The method of claim 58, wherein the formulation comprises red tart cherry juice concentrate is provided at about 39.75 g/dose, Whey protein or whey protein peptide at about 3.15 g/dose, xanthan gum at about 0.08 g/dose, natural cherry pomegranate flavor at about 0.68 g/dose, monk fruit juice concentrate at about 0.11 g/dose, natural masking flavor at about 0.66 g/dose, and citric acid at about 0.12 g/dose.

77. (canceled)
